(19) United States
(12) Patent Application Publication
(10) Pub. No.: US 2020/0398015 A1
(43) Pub. Date: Dec. 24, 2020

(54) COMPENSATED SPLIT VENTILATOR CIRCUIT

(71) Applicant: Keysight Technologies, Inc., Santa Rosa, CA (US)

(72) Inventors: Chee Foo Lum, Bayan Lepas (MY); Hock Chye P'Ng, Tanjung Bungah (MY); Keng Leong Loo, George Town (MY); Phil Stearns, Colorado Springs, CO (US)

(21) Appl. No.: 17/012,639

(22) Filed: Sep. 4, 2020

(51) Int. Cl. A61M 16/08; A61M 16/20; A61M 16/10

(52) U.S. Cl. CPC A61M 16/0833; A61M 16/204; A61M 16/1065; A61M 16/107; A61M 16/205

(57) ABSTRACT

In response to the global shortages of medical ventilator due to emergence of COVID-19 pandemic, the expansion of ventilator availability can be realized by having a Compensated Split Ventilator Circuit fitted into a mechanical ventilator. Hence, a single ventilator may be shared by two patients simultaneously with moderately different lung resistances and compliances. The Compensated Split Ventilator Circuit enables for differential gas volume between two patients by adjusting the valves in the inspiratory and expiratory circuits to control the optimized amount of gas flow to each patient individually. Embodiments of the present invention may be implemented using off-the-shelf medical-grade components to ensure product durability and patient safety.

### BACKGROUND

A ventilator is a device that assists a patient's breathing by providing positive ventilation pressure during surgery or when the patient cannot breathe on his or her own due to a critical illness. Presently available mechanical-based ventilators typically include a single inspiratory and expiratory circuit path for operation with a single patient. During pandemics, such as the current worldwide COVID-19 pandemic, global shortages of ventilators present challenges to the medical community as a result of unprecedented, large numbers of hospital and intensive care unit (ICU) patient admissions, wherein a significant number of the patients require mechanical ventilation for prolonged periods of time.

Because ventilators are technically complex and expensive to produce, on-going attempts to increase production of ventilators have been challenging. Example publications that disclose techniques to increase the patient capacity of ventilators include the following:

Greg Neyman, Charlene Babcock Irvin, “A single ventilator for multiple simulated patients to meet disaster surge”, Department of Emergency Medicine, St. John Hospital and Medical Center, Academy Emergency Medicine, Epub 2006 Aug. 2.
Lorenzo Paladino, Mark Silverberg, Jean G Charchaflieh, Julie K Eason, Brian J Wright, Nicholas Palamidessi, Bonnie Arquilla, Richard Sinert, Seth Manoach, “Increasing ventilator surge capacity in disasters: ventilation of four adult-human-sized sheep on a single ventilator with a modified circuit”, Department of Emergency Medicine, State University of New York Downstate Medical Center, Epub 2007 Dec. 31.
Columbia University Vagelos College of Physicians and Surgeons New York-Presbyterian Hospital “Ventilator Sharing Protocol: Dual-Patient Ventilation with a Single Mechanical Ventilator for Use during Critical Ventilator Shortages”, version 5; Apr. 2, 2020 (Retrieved from protocol s.nyp.org)
Chatburn, R. L., Branson, R. D., & Umur, H. “Multiplex Ventilation: A Simulation-Based Study of Ventilating 2 Patients with a Single Ventilator”. Respiratory Care, 920-931; 2020 Jul. 1.

### Herrmann, J., da Cruz, A. F., Hawley, M. L., Branson, R. D., & Kaczka, D. W. “Shared Ventilation in the Era of COVID-19: A Theoretical Consideration of the Dangers and Potential Solutions”; Respiratory Care, 932-945; 2020.

Tonetti, T., Zanella, A., Pizzilli, G., & at. al. “One ventilator for two patients: feasibility and considerations of a last resort solution in case of equipment shortage”. Thorax, 75: 517-519; 2020. (Retrieved from Thorax: http://dx.doi.org/10.1136/thoraxjn1-2020-214895)

Some of these published “sharing” techniques for ventilators disclose using a single ventilator with multiple patients by applying equal ventilation dynamics to the multiple patients throughout respiratory cycles of the patients. Efficient ventilation of different patients at the same time may be difficult because the patients typically have dissimilar lung dynamics due to pathological variations such as Acute Respiratory Distress Syndrome (ARDS) or Differing Patient Lung Variables (DPLV).

Other published ventilator sharing techniques also enable two or more patients to be connected to one ventilator. However, while ventilator sharing may increase the patient capacity, prior art ventilator sharing techniques may result in ventilator-patient dyssynchrony; cross-infection via inter-patient gas exchange; inability to set the individual patient's tidal volume, oxygen concentration, positive end-expiratory pressure, and difficulty of monitoring an individual patient's tidal volume, flow and pressure.

Accordingly, there is a need to increase ventilator capacity while reducing some of the shortcomings of presently disclosed ventilator sharing techniques.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 shows a standard configuration of a ventilator for a single patient.

FIG. 2 shows a ventilator system including the ventilator and a Compensated Split Ventilator Circuit (200) according to embodiments of the present invention.

FIG. 3 shows construction of the Compensated Split Ventilator Circuit according to embodiments of the present invention.

FIG. 4 shows an example parts list for construction of the Compensated Split Ventilator Circuit according to embodiments of the present invention.

FIG. 5 shows a schematic connection of Compensated Split Ventilator Circuit according to embodiments of the present invention, wherein the ventilator is shown supporting two patients.

FIG. 6 shows a flowchart of an example safety test for the Compensated Split Ventilator Circuit and the ventilator.

### DETAILED DESCRIPTION

Embodiments of the ventilator system 500 include a Compensated Split Ventilator Circuit 200 comprising an inspiratory gas volume splitter 201, an expiratory gas volume splitter 202, pneumatic (PEEP) valves 203-206, gauges (manometer) 207, 208 and bacterial/viral filters (HEPA) 209, 210 that are adapted for use in a standard mechanical ventilator 100. The Compensated Split Ventilator Circuit 200 enables a single ventilator to support two patients simultaneously, even when patients have dissimilar physiological attributes and dissimilar pathological factors.

Frequently used abbreviations associated with embodiments of the present invention may be used where applicable.
ARDS: Acute Respiratory Distress Syndrome
COVID-19: Corona Virus Disease 2019
DPLV: Differing Patient Lung Variables
HEPA filter: High-Efficiency Particulate Air filter
HME filter: Heat and Moisture Exchanger filter
PEEP: Positive End-Expiratory Pressure
PIP: Peak Inspiratory Pressure
V_{T}: Tidal Volume

Integrating the Compensated Split Ventilator Circuit 200 into the ventilator 100 in the ventilator system 500 may expand the use capacity of the ventilator, for example, during emergencies when a large number of patients causes ventilator shortages. The single ventilator 100 when shared by two patients using the Compensated Split Ventilator Circuit 200 may provide compliance with the clinical protocol titled “Ventilator Sharing Protocol: Dual-Patient Ventilation with a Single Mechanical Ventilator for Use during Critical Ventilator Shortages” (version 5; Apr. 2, 2020) depicting a shared ventilator strategy for COVID-19 patients with Acute Respiratory Distress Syndrome.

FIG. 1 shows a schematic of ventilator 100 for use with one-patient in a conventional prior art configuration. The ventilator 100 typically includes a gas source 101 that provides respiratory gas (alternatively referred to as “air) that travels through an inspiratory valve 103 to a humidifier 105. The humidifier 105 includes a heated water reservoir to heat the respiratory gas to reduce patient risks of hypothermia, disruption of the airway epithelium, bronchospasm, atelectasis and airway obstruction. Air that is “loaded” with water vapor in the reservoir travels along an inspiratory circuit 110 to the airway of the patient 300. The exhaled gas from the airway of the single patient 300 then travels along an expiratory circuit 112, passing through a filter 108 to prevent contamination before reaching back to the ventilator's expiratory valve 104. The gas flow that exits from the expiratory valve 104 is detected by sensors 102 for operational monitoring.

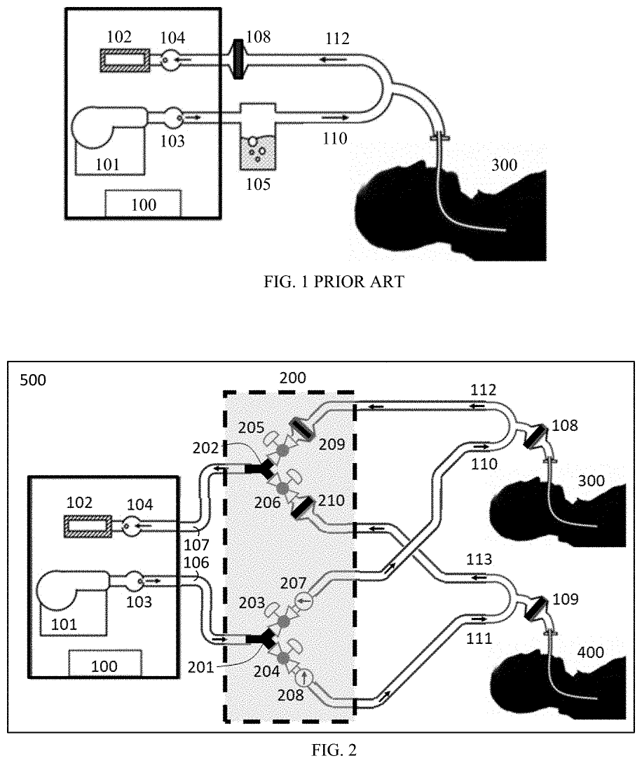

FIG. 2 shows the ventilator system 500 comprising the Compensated Split Ventilator Circuit 200 coupled to the ventilator 100 for ventilation of lungs of two patients 300, 400, with independent selective application of inspiratory pressure and positive end-expiratory pressure for both patients. The Compensated Split Ventilator Circuit 200 comprises two isolated inspiratory pathways or circuits 110, 111 and two isolated expiratory pathways or circuits 112, 113 implemented using Y-Splitters 201, 202. The common node of the Y-Splitter 201, 202 of the inspiratory circuits 110, 111 is connected to breathing tube 106 and the common node 202 of the expiratory circuits 112, 113 is connected to breathing tube 107. The breathing tubes 106, 107 corresponding to each of the patients 300, 400, are generally known as endotracheal tubes and may be physically distinguished using colored tape, different colored tubes, labels or other suitable means.

To reduce cross-contamination between the two patients 300, 400 on the shared-ventilation system, an HME filter 108, 109 is attached proximal to the patients 300, 400, after the manometer gauges 207, 208 based on the directional flow of the gas. In addition, HEPA filters 209, 210 are attached to the expiratory circuits 112, 113 distal from the patients 300, 400, before the expiratory PEEP valves 205, 206 based on the directional flow of the gas, to protect the ventilator 100 from patient contamination. The use of in-line, unidirectional PEEP valves 203, 204, 205, 206 on the inspiratory circuits 110, 111 and expiratory circuits 112, 113 may reduce the likelihood of infections, viruses or bacteria from the patients 300, 400 travelling upstream of the inspiratory circuits 110, 111 and may prevent contamination of the inspiratory valve 103 and expiratory valve 104 and the ventilator 100.

The ventilator system 500 can also control the current volume of gas supplied to the lungs of each of the patients 300, 400 independently by adjusting the corresponding unidirectional PEEP valves 203, 204 that divide the main gas flow in the inspiratory circuits 110, 111. Therefore, by controlling the unidirectional PEEP valves 203, 204 individually, the inspiratory pressure delivered to each of the patients 300, 400 can be reduced or increased as required. The inspiratory pressure may also be measured by reusable dual in-line manometer gauges 207, 208. Adjusting the PEEP valves 203, 204 helps to independently and separately balance the needs of patients 300, 400 that have different and evolving lung impedance (resistance), making the gas volume division for each patient 300, 400 to accommodate variations of mechanical parameters, such as resistance and compliance of the lungs of the patients 300, 400. Resistance is related to conditions like asthma, where there is a constriction of the airway, which can limit (or resist) the flow of air. Increasing the inspiratory pressure increases the volume of air forced through that resistance. On the other hand, compliance is more related to conditions like Chronic Obstructive Pulmonary Disease (COPD), which can impact the plasticity of lungs, and their ability to return to their initial state. This can impact how long oxygen is held in the lungs and absorbed into the blood stream.

A complementary adjustment to the expiratory circuits 112, 113, enables the ventilator system 500 to be customized for the patient's PEEP level using a second pair of in-line unidirectional PEEP valves 205, 206. The PEEP valves 205, 206 deliver additive PEEP to supplement the setting of the ventilator 100 to balance between the patients 300, 400 having different and evolving lung compliance. Adjustment of the valves 203, 204 on the inspiratory circuits 110, 111 and the valves 205, 206 on expiratory circuits 112, 113, may also compensate for these changes between the respective patient(s) 300, 400.

As the Compensated Split Ventilator Circuit 200 allows for the increment and reduction of PIP and PEEP through the use of PEEP valves 203, 204, 205, 206, care must be taken to ensure the correct valves are adjusted for the respective patients connected to the ventilator 100 fitted with Compensated Split Ventilator Circuit 200.

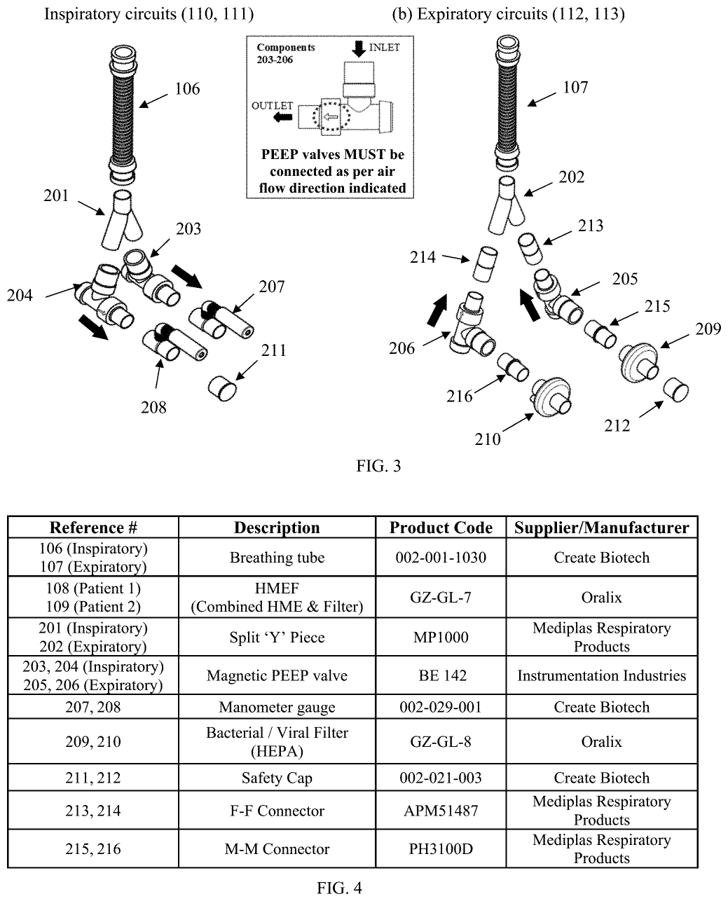

In FIG. 3, the F-F (female-female) connectors 213, 214 and M-M (male-male) connectors 215, 216 are added to enable the connection between the actual parts of the expiratory circuits 112, 113 to match inlet and outlet type/size. For example, the male connections of PEEP valves 205, 206 and male connections of the Y-splitter 202 prohibit mating, then F-F connectors 213, 214 may be added to enable the connection between these components of the Compensated Split Ventilator Circuit 200. In another example, if the PEEP valves 205, 206 and HEPA filters 209, 210 both have female connectors, then M-M (male-male) connectors 215, 216 may be added to enable the connection between components.

For flexibility, the ventilator 100 fitted with Compensated Split Ventilator Circuit 200 in the ventilator system 500 may be easily converted back to single patient use by simply disengaging and removing one of the patient's side tube and replacing the tube with safety caps 211, 212 shown in FIG. 3. Whenever one of the breathing circuits is not in-use, the safety caps may be installed to close off the corresponding ones of the inspiratory circuits 110 or 111 and expiratory circuits 112 or 113. The ventilator parameters as well as the inspiratory PEEP valves 203, 204 and expiratory PEEP valves 205, 206 may also be adjusted accordingly for the remaining single patient.

FIG. 4 shows a table that lists components used in constructing the Compensated Split Ventilator Circuit 200 of the ventilator system 500, including supplier/manufacturer names and specifications. The ventilator system 500 is typically installed without a patient engaged.

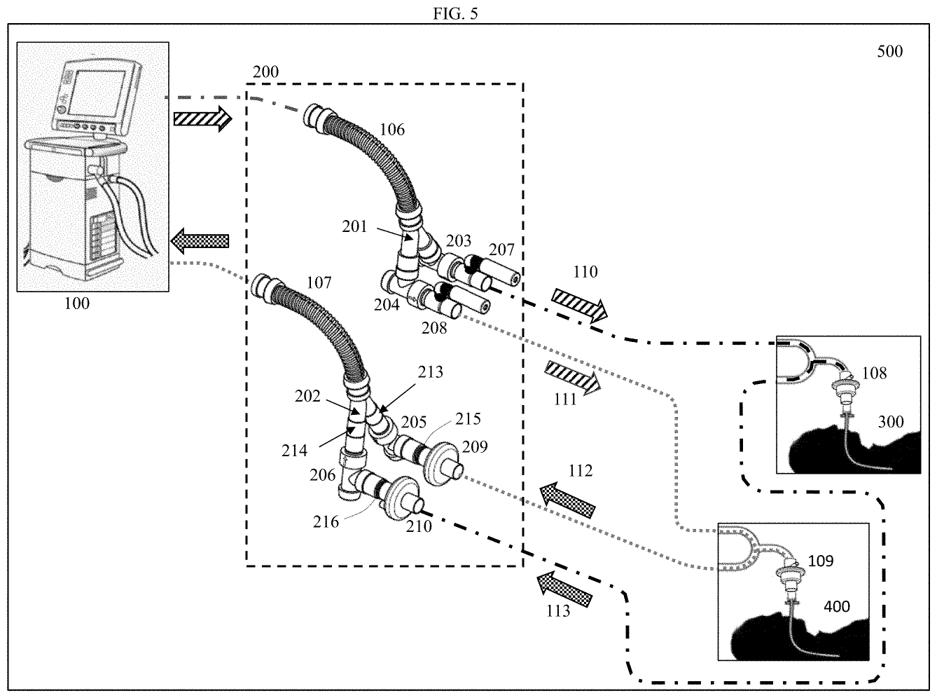

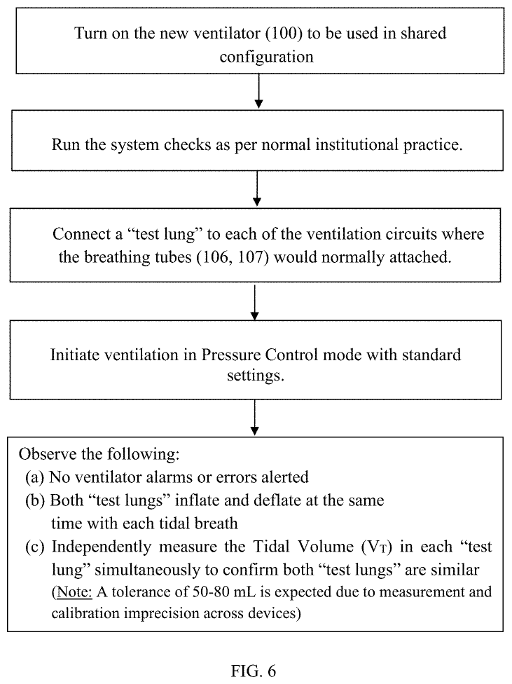

FIG. 5 shows a diagram for connecting the Compensated Split Ventilator Circuit 200 to the ventilator 100 in the ventilator system 500 for support of two patients 300, 400 simultaneously. An example safety test according to the flowchart of FIG. 6 may be carried out before connecting patients to the ventilator system 500.

The present invention has been described herein using specific embodiments for the purpose of illustration only. It will be readily apparent to one of ordinary skill in the relevant art that the principles of the present invention can be embodied in other ways. Therefore, the present invention should not be regarded as being limited in scope to the specific embodiment disclosed herein, but instead as being fully commensurate in scope with the following claims:

1. A ventilation system for simultaneously provisioning inspiratory and expiratory means to two patients, comprising:
a ventilator having an inspiration port providing incoming fresh-gas from a gas source and having an exhalation port;
a parallel pair of inspiratory breathing tubes coupled to the inspiration port of the ventilator through a first Y-splitter, a first of the inspiratory breathing tubes coupled to a first branch of the first Y-splitter and coupled to a first unidirectional valve and a first manometer creating a unidirectional inspiration pathway to a first filter coupled and then to a first patient, a second of the inspiratory breathing tubes coupled to a second branch of the first Y-splitter and coupled to a second unidirectional valve and a second manometer creating a unidirectional inspiration pathway to a second filter coupled to a second patient, whereby the first valve provides a first inspiratory pressure adjustment for the allowable gas flow to the first patient, while the second valve provides a second inspiratory pressure adjustment for the allowable gas flow to the second patient and wherein the first inspiratory pressure adjustment is independent of the second inspiratory pressure adjustment; and the first manometer includes a gauge for measuring an inspiratory pressure in the first of the inspiratory breathing tubes, and wherein the second manometer includes a gauge for independently measuring an inspiratory pressure in the second of the inspiratory breathing tubes; and
a parallel pair of expiratory breathing tubes coupled to the expiration port of the ventilator through a second Y-splitter, a first branch of the second Y-splitter receiving a first expiratory breathing tube coupled the first patient through the first filter, a first HEPA filter and a third unidirectional valve, a second branch of the second Y-splitter receiving a second expiratory breathing tube coupled the second patient through the second filter, a second HEPA filter and a fourth unidirectional valve, whereby the third valve provides for a first adjustment of PEEP pressure to the first patient, and whereby the fourth valve provides for a second adjustment of PEEP pressure to the second patient, wherein the first adjustment is independent of the second adjustment.
